(19) United States
(12) Patent Application Publication
(10) Pub. No.: US 2023/0149089 A1
(43) Pub. Date: May 18, 2023

(54) GUIDANCE OF ARRHYTHMIA ABLATION USING A PATIENT'S HEART DIGITAL TWIN

(71) Applicant: THE JOHNS HOPKINS UNIVERSITY, Baltimore, MD (US)

(72) Inventors: Natalia A. TRAYANOVA, Baltimore, MD (US); Eric SUNG, Baltimore, MD (US); Adityo PRAKOSA, Baltimore, MD (US); Shijie ZHOU, Baltimore, MD (US)

(73) Assignee: THE JOHNS HOPKINS UNIVERSITY, Baltimore, MD (US)

(21) Appl. No.: 17/995,148

(22) PCT Filed: Apr. 6, 2021
(86) PCT No.: PCT/US2021/026035

§ 371 Date: Sep. 30, 2022

Related U.S. Application Data
(60) Provisional application No. 63/005,820, filed on Apr. 6, 2020.

(51) Int. Cl. A61B 34/10; A61B 18/14; G16H 20/40

(52) U.S. Cl. CPC A61B 34/10; A61B 18/1477; G16H 20/40; A61B 2034/105; A61B 2034/104; A61B 2034/107; A61B 2018/00577

(57) ABSTRACT

A method for guiding ablation of atrial or ventricular arrhythmia in a patient's heart is provided. A digital representation of the electrical functioning of atria or ventricles of the patient's heart is generated based on imaging data of the patient's heart that reveals the presence of adipose tissue. The arrhythmias arising in the presence of the adipose tissue in the digital representation of the patients atria or ventricles are determined. The method further includes identifying, in the digital representation, ablation targets that need to be ablated to terminate determined arrhythmias; executing, in the digital representation, a mock-up of a clinical ablation procedure of the patient to determine the electrical response of the patients heart to ablating the ablation targets, and to determine whether the heart continues to generate new arrhythmias post-procedure; and generating a final set of ablation targets based on the mock-up of the clinical ablation procedure.

### CROSS-REFERENCE OF RELATED APPLICATION

This application claims priority to U.S. Provisional Application No. 63/005,820, filed Apr. 6, 2020, the entire contents of which are hereby incorporated by reference.

This invention was made with government support under grant number HL 142893 awarded by the National Institutes of Health/NIH/DHHS. The government has certain rights in the invention.

### BACKGROUND

### 1. Technical Field

Some embodiments relate to systems and methods for subject-specific modeling and digital representations of the subject's heart having adipose tissue (fat).

### 2. Discussion of Related Art

Infiltrating adipose tissue (inFAT) is a newly recognized pro-arrhythmic substrate for post-infarct ventricular tachycardias (VT) that can be identified on contrast-enhanced computed tomography (CE-CT). Fat infiltration in the heart wall is in the form of adipose tissue.

Post-infarct infiltrating adipose tissue (inFAT) has recently been implicated as a pro-arrhythmic substrate that may contribute to life-threatening ventricular tachycardias (VT) [1-4]. inFAT localizes to the infarct [5, 6], develops at a similar timescale as post-infarct VTs [7], and correlates spatially with critical VT circuit sites [1,2]. Catheter ablation, a mainstay of VT treatment [8], disrupts these critical VT circuits with modest success rates in part due to an incomplete understanding of the patient's arrhythmic substrate [9, 10]. inFAT, identifiable on contrast-enhanced computed tomography (CE-CT) [7, 11, 12], may yield valuable information about a patient's substrate to help guide pre-procedural planning and improve ablation efficacy.

Currently, late-gadolinium enhanced cardiac magnetic resonance imaging (LGE-CMR) is the gold standard for pre-procedural substrate assessment [13]. A fibrotic substrate identified on LGE-CMR can be used to identify conduction channels [14, 15] and incorporated into virtual-heart technology to guide ablation targeting [16]. Unfortunately, LGE-CMR is difficult to obtain in clinical practice and has inconsistent image quality due to various artifacts [17]. CT, more accessible than LGE-CMR, has consistent high-resolution image quality for patients with and without implantable cardiovert defibrillator (ICD)s [17] and can be used to characterize substrates including inFAT [18, 19].

What is needed is a “virtual-heart” methodology that incorporates inFAT (adipose tissue) from CE-CT to predict the location of critical VT circuits.

### SUMMARY

According to some embodiments, a computer implemented clinical method for guiding ablation of atrial or ventricular arrhythmia in a patient's heart is provided. A digital representation of the electrical functioning of atria or ventricles of the patient's heart is generated based on imaging data of the patient's heart that reveals the presence of adipose tissue. The arrhythmias arising in the presence of the adipose tissue in the digital representation of the patient's atria or ventricles are determined. The method further includes identifying, in the digital representation, ablation targets that need to be ablated to terminate determined arrhythmias; executing, in the digital representation, a mock-up of a clinical ablation procedure of the patient to determine the electrical response of the patient's heart to ablating the ablation targets, and to determine whether the heart continues to generate new arrhythmias post-procedure; and generating a final set of ablation targets based on the mock-up of the clinical ablation procedure.

According to some embodiments, the computer implemented method may further comprise importing, as part of an ablation procedure of the patient, the final set of ablation targets together with a number of anatomical landmarks from the digital representation into a clinical three-dimensional electroanatomical mapping system in a procedure room of an ablation procedure. According to some embodiments, the method may further comprise registering the imported final set of ablation targets and the imported landmarks to a heart coordinate system of the patient in the clinical three-dimensional electroanatomical mapping system in the operating room during the ablation procedure. According to some embodiments, the computer implemented method may further comprise displaying the generated final set of ablation targets overlaid over an image of the patient's heart in a clinical electroanatomical mapping system in an operating room during an ablation procedure, and navigating an ablation catheter to the final ablation targets.

According to some embodiments the patient imaging data comprises computed tomography (CT) data. According to some embodiments the CT data comprises three-dimensional CT data. According to some embodiments the adipose tissue is one of infiltrating the atrial or ventricular wall, or is epicardial, or pericardial tissue. According to some embodiments the adipose tissue is in combination with fibrosis tissue. According to some embodiments the generating a digital representation of electrical functioning of atria or ventricles of the patient's heart is further based on clinical or experimental data for a regional electrical behavior of cardiac tissue in the presence of adipose tissue. According to some embodiments, the ablation procedure comprises one of endocardial, epicardial or intramural needle ablation to access intramyocardial ablation targets. According to some embodiments, when the digital representation continues to generate arrhythmias after ablation of predicted ablation targets in the mock-up of the clinical procedure, determining any new arrhythmias which arise in the ablated digital representation of the patient's atria or ventricles with adipose tissue, and wherein when the new arrhythmias arise, generating additional ablation targets, and adding the additional ablation targets to a set of initial ablation targets.

According to some embodiments, the determining whether any new arrhythmias arise, and the generating additional ablation targets is repeated, until no new arrhythmias are generated, and the final set of ablation targets is then generated. According to some embodiments, the determining whether any new arrhythmias arise comprises delivering pacing to a number of pacing locations of the digital representation of the patient's atria or ventricles. According to some embodiments, the generating a digital representation comprises creating a finite element mesh using the imaging data of the patient's heart that reveals the presence of adipose tissue, the finite element mesh comprising a plurality of volume elements, wherein the volume elements each represent a volume having an edge length in a range of about 300-400 microns. According to some embodiments a number of the volume elements is greater than one million. According to some embodiments the number of the volume elements is greater than two million.

According to some embodiments, a system corresponding to the method is described.

### BRIEF DESCRIPTION OF THE DRAWINGS

Further objectives and advantages will become apparent from a consideration of the description, drawings, and examples.

FIG. 1 shows a Digital-heart Identification of Fat-based Ablation Targets (DIFAT) workflow.

FIG. 2A shows the quantification of inFAT distributions across 29 patient hearts in a retrospective study.

FIG. 2B shows examples of inFAT distributions in 4 different patients.

FIG. 3 shows examples of critical VT circuits within inFAT.

FIG. 4A shows the distribution of overlapping ablations between digital-heart and clinical ablations across anatomical regions.

FIG. 4B shows examples of co-localization for 8 patients with overlapping ablations

FIG. 5 shows examples of three patients who underwent a redo ablation procedure.

### DETAILED DESCRIPTION

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The below-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. The references cited anywhere in this specification are hereby incorporated by reference as if each had been individually incorporated.

According to some embodiments, there is presented a novel CT-based digital-heart technology for VT ablation targeting in ischemic cardiomyopathy patients based on personalized assessment of the arrhythmogenic substrate arising from presence of inFAT. This approach is termed Digital-heart Identification of Fat-based Ablation Targets (DIFAT). The major advantages of this approach is that it uses easily accessible contrast-enhanced computed tomography (CE-CT) as the imaging modality, making it generalizable to clinical centers without LGE-CMR expertise. Secondly, this approach can anticipate the potential arrhythmogenic effects of ablation lesions on the patient-specific ventricular substrate. Lastly, this approach produces ablation targets that can be readily imported into contemporary electroanatomic mapping systems (EAMs). This approach can be easily integrated into clinical workflows to achieve therapeutic precision of VT ablation targeting and mitigate the need for redo ablation procedures in VT recurrences.

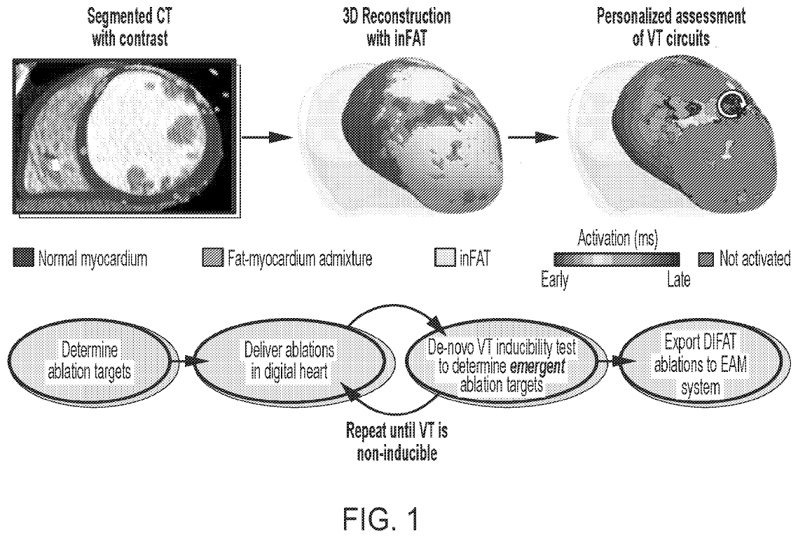

FIG. 1 shows the Digital-heart Identification of Fat-based Ablation Targets (DIFAT) workflow of some embodiments. As shown in the top row of FIG. 1, from CT images with contrast, the myocardium is segmented into non-injured myocardium and inFAT, and fat-infiltrated myocardium is identified. Personalized 3D digital hearts are reconstructed from the segmented data and electrophysiological information. inFAT-based arrhythmogenic propensity is assessed to identify all possible VTs the substrate can sustain. The circular arrow represents the direction of reentrant propagation.

As shown in the bottom row of FIG. 1, in some embodiments all VTs are analyzed to determine ablation targets. These ablation lesions are then incorporated in the digital hearts as a mock-up of the clinical procedure. VT inducibility in the post-ablation digital hearts is tested again to determine whether emergent VTs arise post-ablation. This process is repeated until no VTs can be induced. The finalized DIFAT ablations are exported into an EAM system to guide the ablation procedure.

Abbreviations: DIFAT: Digital-heart Identification of Fat-based Ablation Target; inFAT=infiltrating adipose tissue; VT: ventricular tachycardia, EAM: electroanatomic mapping.

Some embodiments are described in the following examples.

### Examples

Methods Summary: The predictive capabilities of the DIFAT VT ablation technology for post-infarction patients was assessed in 29 patients that had undergone VT ablation. CE-CTs were acquired for all patients prior to their index (i.e. first) ablation procedure. Personalized 3D digital heart models incorporating the patient-specific inFAT distributions were reconstructed. The inFAT's arrhythmogenic propensity in the digital hearts was assessed via rapid pacing from multiple bi-ventricular locations. From the analysis of the induced VTs, DIFAT ablation targets were determined. Next, these targets were implemented as non-conductive lesions in the digital hearts to determine whether the DIFAT ablations the post-infarct ventricular substrate rendered it non-inducible for VT. To assess VT non-inducibility, the VT induction protocol by rapid pacing from a number of ventricular sites was repeated. If new VT arose, the new VT ablation targets were determined, and the procedure was repeated until complete VT non-inducibility is achieved in the post-infarction ventricular substrate.

In the comparison between DIFAT and clinical targets to demonstrate co-predictive capability, electroanatomic map surfaces and ablation locations were registered, using a number of heart anatomical landmarks from CT, to digital hearts.

Results Summary: In the retrospective study of 29 patients, inFAT localized primarily to the apex (0.25% of myocardium volume) and less to the septum (0.04% of myocardium volume). VTs were induced in 28/29 digital hearts. The distribution of VT morphologies induced in digital and patient hearts was similar. Digital-heart ablations co-localized with clinical ablations in 23 out of 29 patients. The overlap occurred primarily in the apex (72.0+/−8.63%) and inferior/inferolateral regions (73.9+/−7.84%), but not in the septum (1.96+/−1.22%). Lastly, the virtual hearts incorporating inFAT revealed new VT circuits that coincided with redo ablation targets performed years after the index ablation.

Methods

Study Population

Data from 29 patients with ischemic cardiomyopathy was used in this study. Inclusion criteria included history of myocardial infarction (MI) and VT, a CE-CT obtained within 1 month of their index ablation procedure, and an ablation procedure performed using the Biosense Webster 3D electroanatomical mapping system. For patients with multiple images, CE-CT was used, obtained from the time of the earliest ablation procedure.

CT Image Acquisition

Cardiac CE-CTs were acquired using a commercially available Toshiba Aquillion 320-detector CT scanner. All scans were performed with prospective ECG-gating at 75-80% of the R-R interval to minimize motion artifact. The tube voltage was 100 kV or 120 kV and tube amperage ranged from 300 to 700 mA, depending on body habitus and heart rate. Iodinated IV contrast was administered intravenously at a rate of 5-6 cc/second with doses from 70-120 mL. Triggering was set to 0.75-1.19 seconds after the intensity in the descending aorta exceeded 200 Hounsfield Units (HU). Scans were reconstructed at an in-plane resolution of 0.428-0.625 mm by 0.428-0.625 mm and slice thickness of 0.5-3 mm.

inFAT Identification

CT images were resampled into short axial view at a resolution of 0.35×0.35×0.35 mm. Images were pre-processed by thresholding to minimize the ICD artifact burden. ICD artifact voxels, defined as intensities <−180 HU or ≥250 HU, were removed along with the surrounding regions from analysis. The myocardium was segmented using a semi-automatic algorithm as presented in previous studies [20, 16]. inFAT in the left ventricle is pathologic whereas inFAT in the right ventricle (RV) can be physiologic [11]. Thus, inFAT found in the RV was excluded. inFAT in the range −180 to −50 HU was identified based on data in the literature [2, 4, 21]. Previous studies have included hypoattenuations >−50 HU as part of inFAT. Hypoattenuated voxels with intensities >−50 HU likely represent a mixture between adipose and lean tissues such as myocardium [22]. To account for this difference, tissues from −50 to −5 HU were distinguished separately from inFAT and termed this region “fat-infiltrated myocardium” (admixture of fat and cardiac cells). To assess the distribution of inFAT, the volume of inFAT was computed in each of 4 anatomical regions (the septum, apex, anterior/anterolateral, and inferior/inferolateral). To account for patient heart size variability, the amount of inFAT was computed within each anatomical region as a percentage of the total myocardial volume.

Personalized 3D Digital Heart Model Reconstruction with inFAT

3D heart models (digital hearts) of the post-infarct left ventricle were reconstructed from the segmented myocardium along with the patients' distributions of inFAT and fat-infiltrated myocardium (FIG. 1). The right ventricle was not reconstructed because RV inFAT was excluded from consideration, as it cannot be well identified in CE-CT. To execute simulations, tetrahedral volume meshes, having volume elements of edge length in the interval 300-400 microns, resulting in meshed of over 4 million elements, of each digital heart were generated using the Materialise Mimics software. For the retrospective study of 29 patients, the average mean edge length across meshes was 393.79+/−0.19 μm. The median (interquartile range) number of mesh nodes was 4881325 (1583624) points. The median (interquartile range) number of tetrahedral volume elements was 29507503 (9643294). The choice of finite element size was dictated by the need to resolve wavefront propagation in the simulations.

To account for conduction anisotropy, realistic myocardial fiber orientations were generated using a previously validated rule-based method [27]. This fiber orientation methodology uses the Laplace-Dirichlet method to define transmural and apicobasal directions at every point in the patient-specific ventricles. It then employs bi-directional spherical linear interpolation to assign fiber orientations based on a set of fiber orientation properties (rules).

Assigning Electrophysiology Properties in the inFAT Digital Hearts

inFAT was modeled as a non-conducting insulator region. The electrophysiological effects of inFAT on surrounding ventricular tissue are poorly understood. Evidence suggests that gap junction remodeling [18], decreased conduction velocity [18,28], and altered electrogram signals [19,21] take place in fat-infiltrated myocardium, similar to the electrophysiological changes in the infarct border zone [29], however the extent of these changes remains unknown. In the absence of such data, the electrophysiological properties of the fat-infiltrated myocardium were approximated with those of the peri-infarct zone, the latter presented in detail in our previous publications [9,24]. The normal myocardium conductivity and action potential dynamics were the same as in our previous works [9,24].

VT Simulation of Electrical Activity and Numerical Aspects.

Each mesh node in the finite element mesh was modeled as a myocyte with membrane dynamics described by the system of ordinary differential and algebraic equations describing local electrophysiological ionic properties as described above. To simulate digital-heart electrical activity, a reaction-diffusion partial differential equation, representing myocardium current propagation, was solved, together with the system of ordinary differential equations at each node at the finite element mesh node, using a time step of 25 μs. The equations were solved on a high-performance parallel computing system.

Patient Characteristics in the Retrospective Study

Patient characteristics are summarized in Table 1. All 29 enrolled patients had an implanted ICD. The median age at the time of the first ablation procedure was 63 years with an interquartile range of 12 years. The median ejection fraction was 30% with an interquartile range of 15%, and the median infarct age was 18.5 years with an interquartile range of 11.25 years.

TABLE 1
Baseline characteristics of patients
enrolled in the retrospective study

| | Patients (N = 29) |
|---|---|
| Age, y | 63 (12) |
| Infarct Age, y | 18.5 (11.25) |
| Male | 21 (72.4%) |
| ICD implanted | 29 (100%) |
| LVEF, % | 30 (15) |
| # VTs Induced in Procedure | 3 (2) |

Categorical variables are expressed as the count (percentage). Continuous and ordinal variables are expressed as median (25^{th}-75^{th} Interquartile range).

Abbreviations: VT: ventricular tachycardia, LVEF: left ventricular ejection fraction, ICD: implanted cardioverter defibrillator.

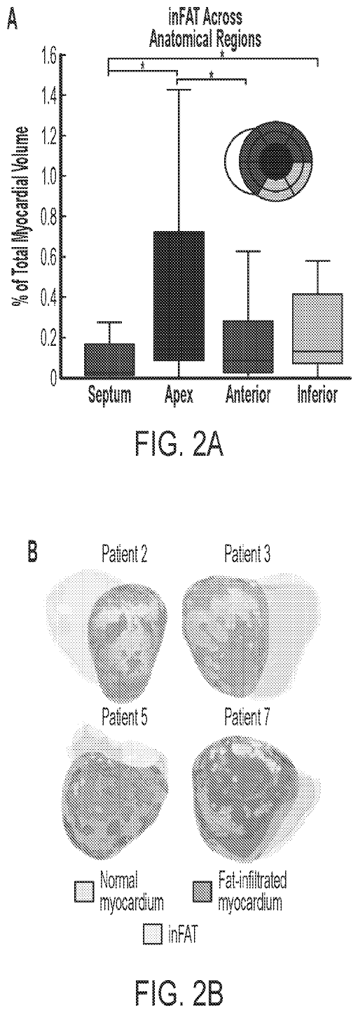

FIG. 2A shows that inFAT localizes primarily to the apex, illustrating the quantification of inFAT across all patient hearts. Box plots denote inFAT percentage of myocardium volume. There was a significant difference in inFAT across anatomical regions (p<0.001). There was more inFAT in the apex than in the anterior/anterolateral region (p<0.05). There was significantly less fat inFAT in the septum than in the apex and inferior/inferolateral regions of the heart (p<0.05).

inFAT and fat-infiltrated myocardium was present to varying degrees in all patients with a median of 0.81% (IQR: 1.25%) for inFAT and 3.02% (IQR: 3.27%) for fat-infiltrated myocardium. The distributions of inFAT across anatomical regions are summarized in FIG. 2A. The most amount of inFAT was found in the apical regions, and the least amount of inFAT was found in the septum (FIG. 2A). There was a significant difference between the inFAT distributions in the 4 anatomical regions (p<0.001). The median inFAT percentage was 0.04% in the septum, 0.25% in the apex, 0.09% in the anterior/anterolateral regions, and 0.14% in the inferior/inferolateral regions. Patients had more inFAT in the apex than the anterior/anterolateral regions (p<0.05). Patients had less inFAT in the septum than in the apex (p<0.05) and inferior/inferolateral regions (p<0.05) (FIG. 2A).

FIG. 2B shows that inFAT distribution was highly variable across patients, with examples of inFAT distributions in 4 different patients. inFAT was present in the anterior region and apex (patient 2, top left), the inferior region (patient 3, top right), the anterolateral region (patient 5, bottom left), and inferolateral region (patient 7, bottom right).

FIG. 2B illustrates the widely varying patterns of inFAT distribution across patients. For instance, patient 2 had inFAT covering much of the apical and anterior regions whereas patient 3 had inFAT spanning much of the inferior region. Unlike patients 2 and 3, patient 5 did not have a continuous distribution of inFAT and fat-infiltrated myocardium, but rather had a patchier distribution in the lateral and anterolateral regions. Lastly, patient 7's inFAT distribution was also patchy with a variable extent of inFAT penetration into the mid-myocardium in the lateral and inferolateral regions.

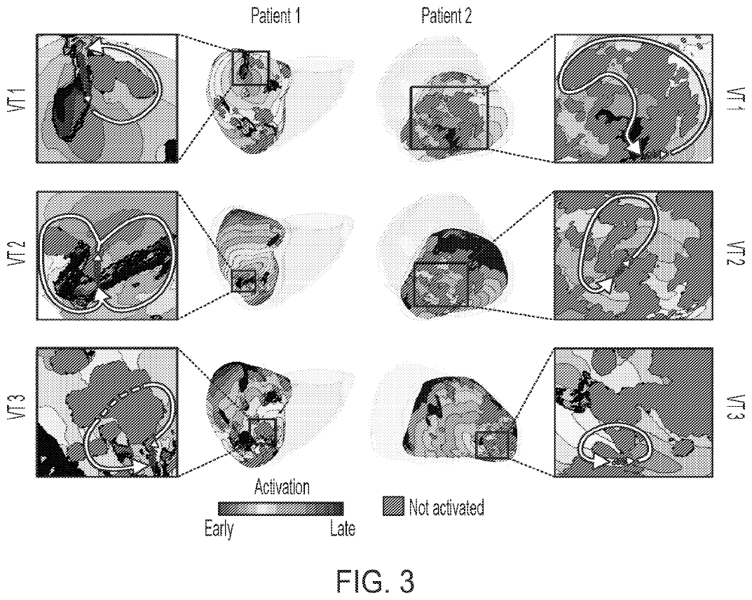

FIG. 3 shows that inFAT forms conduction channels that harbor potential critical VT circuits. Several examples of critical VT circuits within inFAT are shown, for patients 1 and 2 who both had 3 VT morphologies (activation maps shown). Gray denotes non-activated tissue; red is the earliest activation and dark blue is the latest activation. The circular white arrow traces the re-entrant pathway.

In 28 out of 29 patients, DIFAT uncovered conduction channels formed by the patient-specific inFAT distribution. For patient 1, DIFAT identified three distinct VT circuits. The inFAT and mitral annulus formed inexcitable obstacles, facilitating formation and maintenance of VT 1 (FIG. 3, top left). Both VT2 and VT3 propagated through conduction channels formed by inFAT and fat-infiltrated myocardium in the mid inferior (FIG. 3, middle and bottom left). For patient 2, there was extensive inFAT in the anterior region and apex which harbored three VT circuits. VT1 had a macro-reentrant pathway that spanned across much of the inFAT penetrated region (FIG. 3 top right). VT2 was located within the center of the inFAT in the apical lateral region (FIG. 3, middle right), and VT3 localized to the periphery of the inFAT in the apical septum (FIG. 3, bottom right).

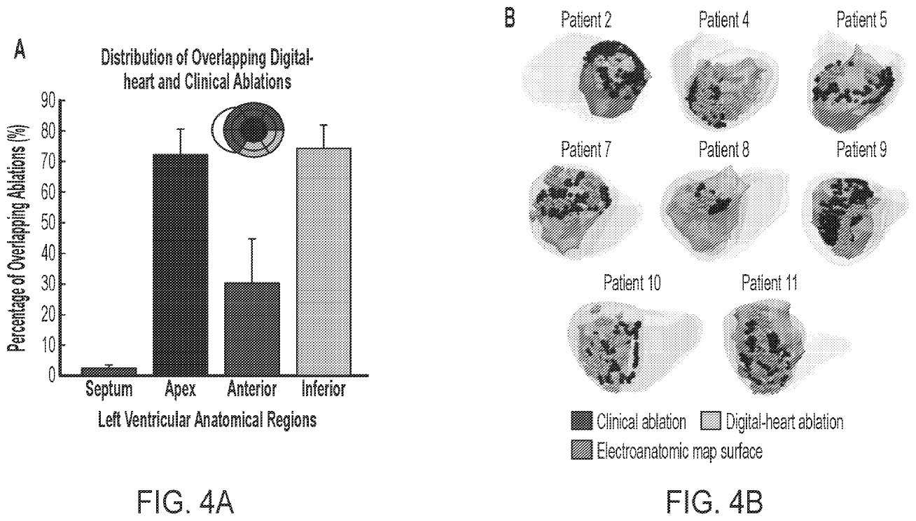

FIG. 4A shows the distribution of overlapping ablations between digital-heart and clinical ablations across anatomical regions. DIFAT ablations co-localized with clinical ablations for 23 out of the 29 patients. The overlap primarily occurred in the apex and inferior/inferolateral regions, but not the septum (left). The DIFAT and clinical ablations primarily overlapped in the apex (72.0+/−8.63%) and inferior/inferolateral regions (73.9+/−7.84%), partially overlapped in the anterior/anterolateral region (30.1+/−14.6%) and did not overlap well in the septum (1.96+/−1.22%) (FIG. 4A).

FIG. 4B shows examples of co-localization for 8 patients with overlapping ablations. Dark red denotes clinical ablation lesions, orange denotes digital-heart ablations, and light blue denotes the electroanatomical (EAM) surface. Overlapping ablations primarily localized to the apex for patients 2 and 4, the anterior/anterolateral region for patient 5, and the inferior/inferolateral regions for patients 7 and 8. (FIG. 4B). Lastly, patients 9, 10, and 11 had more extensive clinical ablations spanning both the inferior/inferolateral regions as well as the apex. For these three patients, the overlapping ablations occurred in both inferior/inferolateral regions and the apex, again highlighting DIFAT's predictive capabilities. These results suggest that DIFAT consistently predicts ablation targets for post-infarct VTs originating from various locations in the left ventricle.

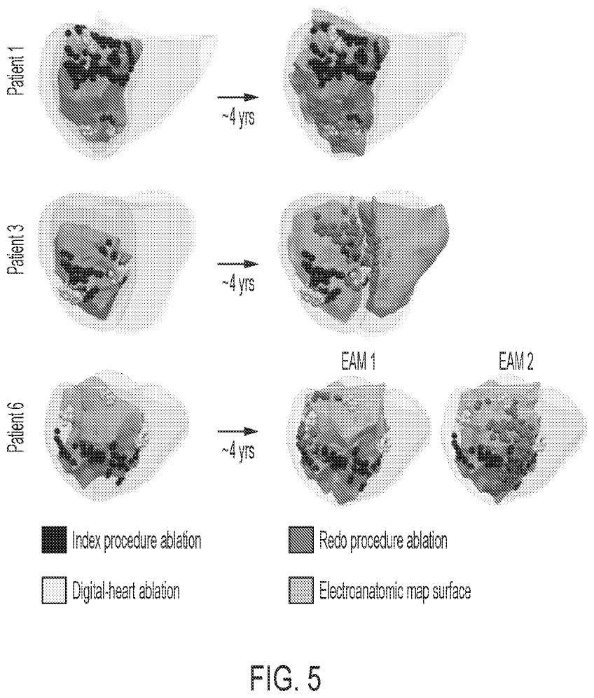

In some embodiments, DIFAT predicts VT recurrence and/or emergence, which is a capability that is unique to DIFAT. FIG. 5 shows examples of three patients who underwent a redo ablation procedure roughly 4 years after their index ablation procedure. First procedure ablations are illustrated on the left side of FIG. 5. First procedure clinical ablations co-localized well with sets of virtual-heart ablations for patients 1 and 3. Repeat procedure ablations shown with first procedure ablations are illustrated on the right side of FIG. 5. Repeat ablations are shown in magenta. Repeat procedure ablations co-localized well with digital-heart ablations for patients 1, 3, and 6. DIFAT was able to predict regions that would be ablated in the repeat procedure. For all 3 cases, DIFAT predicted new VT circuits that were targeted in redo ablation procedures. 10 out of the 29 patients had more than one VT ablation procedure separated at least a month apart. Of these patients with VT recurrence, 7 patients had repeat ablations performed in the same location as in a prior procedure. The remaining 3 patients (1, 3, and 6) had clearly distinct ablation sites delivered in a redo ablation procedure about 4 years after the index procedure (FIG. 5).

For these 3 patients, the location of index and redo procedure ablations were compared with the virtual-heart ablations, shown in FIG. 5. For patient 1, the index ablations were delivered to the basal inferior whereas the redo ablations were delivered to the mid inferior, focusing on VT circuits not targeted in the index procedure. DIFAT predicted 3 VT circuits: one circuit coinciding with the index ablations (FIG. 5, top left) and two new VT circuits that aligned well with the redo procedure ablations (FIG. 5, top right). For patient 3, the left set of DIFAT ablations co-localized with the index ablations (FIG. 5, middle left) whereas the right set of DIFAT ablations coincided with the redo ablations (FIG. 5, middle right), once again demonstrating DIFAT's ability to preemptively predict emergent VT circuit locations. Lastly, for patient 6, the DIFAT ablations only partially co-localized with the index procedure ablations (FIG. 5, bottom left). However, DIFAT's predictions coincided well with both sets of the extensive redo ablations (FIG. 5, bottom right), again demonstrating DIFAT's ability to predict VT recurrences.

Discussion

This study presented DIFAT, a digital-heart technology that incorporates post-infarct inFAT distribution from CT to predict individualized VT ablation targets in ischemic cardiomyopathy patients. DIFAT non-invasively assesses the arrhythmogenic propensity of the inFAT substrate to determine all possible VTs that it can sustain and uses this information to determine the optimal VT ablation targets. DIFAT is designed to completely eliminate the ability of the patient's inFAT substrate to sustain VT. This ablation concept is radically different from any existing VT ablation strategies, with an aim to eliminate not only the clinically manifested VT, but also latent VTs that could arise from the inFAT substrate, including those that might emerge following initial ablation. Finally, DIFAT has a unique advantage in that it identifies inFAT from CT, an imaging resource that is widely accessible, which renders DIFAT implementable in a broad range of centers without LGE-CMR expertise, and for patients with ICDs. DIFAT ablation targets can easily be imported into electroanatomical mapping systems, the methodology for which has been validated in previous studies [9,11]. Implementation of DIFAT could significantly improve the precision of VT ablation therapy and decrease VT recurrence rates, reducing the burden of costly redo ablation procedures.

The present retrospective study assessed the predictive capabilities of the technology by comparing DIFAT ablation targets to clinical ablation data from 29 patients with ischemic cardiomyopathy, all with ICDs. DIFAT predicted a similar number of VT morphologies in the digital hearts as in the patient hearts, suggesting concordance in arrhythmogenicity between them. DIFAT ablations not only co-localized with the index ablations in most cases, but also with the redo ablations, highlighting the ability of DIFAT to predict emergent VT circuits that manifested years later.

### REFERENCES

1. Pouliopoulos J, Chik W W B, Kanthan A, et al. Intramyocardial Adiposity After Myocardial Infarction. Circulation. 2013; 128(21):2296-2308. doi:10.1161/CIRCULATIONAHA.113.002238
2. Sasaki T, Calkins H, Miller C F, et al. New insight into scar-related ventricular tachycardia circuits in ischemic cardiomyopathy: Fat deposition after myocardial infarction on computed tomography—A pilot study. Hear Rhythm. 2015; 12(7):1508-1518. doi:10.1016/J.HRTHM.2015.03.041
3. Samanta R, Pouliopoulos J, Thiagalingam A, Kovoor P. Role of adipose tissue in the pathogenesis of cardiac arrhythmias. Hear Rhythm. 2016; 13(1):311-320. doi:10.1016/j.hrthm.2015.08.016
4. Cheniti G, Sridi S, Sacher F, et al. Post-Myocardial Infarction Scar With Fat Deposition Shows Specific Electrophysiological Properties and Worse Outcome After Ventricular Tachycardia Ablation. doi:10.1161/JAHA.119.012482
5. Baroldi, G. Email Author, Silver, M. D., De Maria, R., Parodi, O., Pellegrini A. Lipomatous Metaplasia in Left Ventricular Scar. https://www.scopus.com/record/display.uri?eid=2-s2.0-0031029585&origin=inward&txGid=6a929687e6b7b03eb9a23d85eb61b39d.
6. Su L, Siegel J E, Fishbein M C. Adipose tissue in myocardial infarction. Cardiovasc Pathol. 2004; 13(2):98-102. doi:10.1016/S1054-8807(03)00134-0
7. Ichikawa Y, Kitagawa K, Chino S, et al. Adipose Tissue Detected by Multislice Computed Tomography in Patients After Myocardial Infarction. JACC Cardiovasc Imaging. 2009; 2(5):548-555. doi:10.1016/j.jcmg.2009.01.010
8. 2019 HRS/EHRA/APHRS/LAHRS Expert Consensus Statement on Catheter Ablation of Ventricular Arrhythmias|Heart Rhythm Society. https://www.hrsonline.org/clinical-resources/2019-hrsehraaphrslahrs-expert-consensus-statement-catheter-ablation-ventricular-arrhythmias. Accessed Nov. 12, 2019.
9. Marchlinski F E, Haffajee C I, Beshai J F, et al. Long-term success of irrigated radiofrequency catheter ablation of sustained ventricular tachycardia: Post-approval THERMOCOOL VT trial. J Am Coll Cardiol. 2016. doi:10.1016/j.jacc.2015.11.041
10. Tanner H, Hindricks G, Volkmer M, et al. Catheter ablation of recurrent scar-related ventricular tachycardia using electroanatomical mapping and irrigated ablation technology: Results of the prospective multicenter Euro-VT-study. J Cardiovasc Electrophysiol. 2010; 21(1):47-53. doi:10.1111/j.1540-8167.2009.01563.x
11. Raney A R, Saremi F, Kenchaiah S, et al. Multidetector computed tomography shows intramyocardial fat deposition. J Cardiovasc Comput Tomogr. 2008; 2(3):152-163. doi:10.1016/J.JCCT.2008.01.004
12. Winer-Muram H T, Tann M, Aisen A M, Ford L, Jennings S G, Bretz R. Computed Tomography Demonstration of Lipomatous Metaplasia of the Left Ventricle Following Myocardial Infarction. J Comput Assist Tomogr. 2004; 28(4):455-458. doi:10.1097/00004728-200407000-00004
13. Markman T M, Nazarian S. Treatment of ventricular arrhythmias: What's New? Trends Cardiovasc Med. 2019; 29(5):249-261. doi:10.1016/j.tcm.2018.09.014
14. Berruezo A, Fernandez-Armenta J, Andreu D, et al. Scar dechanneling. Circ Arrhythmia Electrophysiol. 2015; 8(2):326-336. doi:10.1161/CIRCEP.114.002386
15. Andreu D, Penela D, Acosta J, et al. Cardiac magnetic resonance-aided scar dechanneling: Influence on acute and long-term outcomes. Hear Rhythm. 2017; 14(8):1121-1128. doi:10.1016/j.hrthm.2017.05.018
16. Prakosa A, Arevalo H J, Deng D, et al. Personalized virtual-heart technology for guiding the ablation of infarct-related ventricular tachycardia. Nat Biomed Eng. 2018; 2(10):732-740. doi:10.1038/s41551-018-0282-2
17. Symons R, Zimmerman S L, Bluemke D A. CMR and CT of the Patient With Cardiac Devices: Safety, Efficacy, and Optimization Strategies. JACC Cardiovasc Imaging. 2019. doi:10.1016/j.jcmg.2018.09.030
18. Takigawa M, Duchateau J, Sacher F, et al. Are wall thickness channels defined by computed tomography predictive of isthmuses of postinfarction ventricular tachycardia? Hear Rhythm. June 2019. doi:10.1016/j.hrthm.2019.06.012
19. Cedilnik N, Duchateau J, Dubois R, et al. Fast personalized electrophysiological models from computed tomography images for ventricular tachycardia ablation planning. EP Eur. 2018; 20(suppl 3):iii94-iii101. doi:10.1093/europace/euy228
20. Prakosa A, Malamas P, Zhang S, et al. Methodology for image-based reconstruction of ventricular geometry for patient-specific modeling of cardiac electrophysiology. Prog Biophys Mol Biol. 2014; 115(2-3):226-234. doi:10.1016/j.pbiomolbio.2014.08.009
21. Thanassoulis G, Massaro J M, O'Donnell C J, et al. Pericardial fat is associated with prevalent atrial fibrillation: The framingham heart study. Circ Arrhythmia Electrophysiol. 2010; 3(4):345-350. doi:10.1161/CIRCEP.109.912055
22. Borkan G A, Gerzof S G, Robbins A H, Hults D E, Silbert C K, Silbert J E. Assessment of abdominal fat content by computed tomography. Am J Clin Nutr. 1982; 36(1):172-177. doi:10.1093/ajcn/36.1.172
23. Bayer J D, Blake R C, Plank G, Trayanova N A. A Novel Rule-Based Algorithm for Assigning Myocardial Fiber Orientation to Computational Heart Models. doi:10.1007/s10439-012-0593-5
24. Ustunkaya T, Desjardins B, Liu B, et al. Association of regional myocardial conduction velocity with the distribution of hypoattenuation on contrast-enhanced perfusion computed tomography in patients with postinfarct ventricular tachycardia. Hear Rhythm. 2019; 16(4):588-594. doi:10.1016/j.hrthm.2018.10.029
25. Arevalo H J, Vadakkumpadan F, Guallar E, et al. Arrhythmia risk stratification of patients after myocardial infarction using personalized heart models. Nat Commun. 2016; 7. doi:10.1038/ncomms11437
26. Ten Tusscher K H W J, Panfilov A V. Alternans and spiral breakup in a human ventricular tissue model. Am J Physiol—Hear Circ Physiol. 2006; 291(3). doi:10.1152/ajpheart.00109.2006
27. Verani M S, Kaul S, Laskey W K, Pennell D J, Rumberger J A, Ryan T. American Heart Association Writing Group on Myocardial Segmentation and Registration for Committee of the Council on Clinical Cardiology of the American Heart Association the Heart: A Statement for Healthcare Professionals From the Cardiac Imaging Standardized Myocardial Segmentation and Nomenclature for Tomographic Imaging of 2002. doi:10.1161/hc0402.102975
28. Antz M, Berodt K, Bänsch D, et al. Catheter-ablation of ventricular tachycardia in patients with coronary artery disease: influence of the endocardial substrate size on clinical outcome. Clin Res Cardiol. 2008; 97:110-117. doi:10.1007/s00392-007-0596-7
29. Srinivasan N T, Orini M, Providencia R, et al. Prolonged action potential duration and dynamic transmural action potential duration heterogeneity underlie vulnerability to ventricular tachycardia in patients undergoing ventricular tachycardia ablation. EP Eur. 2019; 21(4):616-625. doi:10.1093/europace/euy260
30. Shirai Y, Liang J J, Santangeli P, et al. Long-term outcome and mode of recurrence following noninducibility during noninvasive programmed stimulation after ventricular tachycardia ablation. doi:10.1111/pace.13605
31. Bhaskaran A, Nayyar S, Porta-Sanchez A, et al. Direct and indirect mapping of intramural space in ventricular tachycardia. Hear Rhythm. October 2019. doi:10.1016/j.hrthm.2019.10.017
32. Stevenson W G, Tedrow U B, Reddy V, et al. Infusion Needle Radiofrequency Ablation for Treatment of Refractory Ventricular Arrhythmias. J Am Coll Cardiol. 2019; 73(12):1413-1425. doi:10.1016/j.jacc.2018.12.070

The above provides examples according to particular embodiments of the current invention. The broad concepts of the current invention are not limited to only those particular examples. More generally, a computer implemented clinical method for guiding ablation of atrial or ventricular arrhythmia in a patient's heart, includes generating a digital representation of electrical functioning of atria or ventricles of the patient's heart based on imaging data of the patient's heart that reveals the presence of adipose tissue. Any arrhythmias arising in the presence of the adipose tissue in the digital representation of the patient's atria or ventricles are determined. In the digital representation, ablation targets that need to be ablated to terminate determined arrhythmias are identified. In the digital representation, a mock-up of a clinical ablation procedure of the patient to determine the electrical response of the patient's heart to ablating the ablation targets, and to determine whether the heart continues to generate new arrhythmias post-procedure are executed. A final set of ablation targets based on the mock-up of the clinical ablation procedure are generated.

The method may further include registering or importing procedures. For example, the method may include importing, as part of an ablation procedure of the patient, the final set of ablation targets together with a number of anatomical landmarks from the digital representation into a clinical three-dimensional electroanatomical mapping system in a procedure room of an ablation procedure. The method may include registering the imported final set of ablation targets and the imported landmarks to a heart coordinate system of the patient in the clinical three-dimensional electroanatomical mapping system in the operating room during the ablation procedure. Thus, the method may provide ablation targets for an ablation procedure.

As another example, the method may include displaying the generated final ablation targets overlaid over an image of the patient's heart in a clinical electroanatomical mapping system in an operating room during an ablation procedure, and navigating an ablation catheter to the final ablation targets.

The patient image data use to generate the digital representation may be broadly provided. The patient image data may include, for example, computed tomography (CT) data. In particular, the patient image data may include three-dimensional CT data. Aspects of embodiments, however, are not limited to CT data as the patient image data.

The presence of adipose tissue in the image data may be of several varieties, for example. The adipose tissue may be one of infiltrating, epicardial, or pericardial tissue, for example. The adipose tissue may be in combination with fibrosis tissue, for example.

Other features of the embodiments should be provided broadly. For example, the particular ablation procedure is not limited. For example, the ablation procedure may include one of endocardial, epicardial or intramural needle ablation to access intramyocardial ablation targets. Further, the generating a digital representation of electrical functioning of atria or ventricles of the patient's heart may be further based on clinical or experimental data for a regional electrical behavior of cardiac tissue in the presence of adipose tissue.

Further, when the digital representation continues to generate arrhythmias after ablation of predicted ablation targets in the mock-up of the clinical procedure, any new arrhythmias which arise in the ablated digital representation of the patient's atria or ventricles with adipose tissue are determined, and when the new arrhythmias arise, additional ablation targets are generated, and additional ablation targets are added to a set of initial ablation targets. The determining whether any new arrhythmias arise, and the generating additional ablation targets may be repeated, until no new arrhythmias are generated, and the final set of ablation targets is then generated.

What is claimed is:

1. A computer implemented clinical method for guiding ablation of atrial or ventricular arrhythmia in a patient's heart, comprising:
generating a digital representation of the electrical functioning of atria or ventricles of the patient's heart based on imaging data of the patient's heart that reveals the presence of adipose tissue;
determining the arrhythmias arising in the presence of the adipose tissue in the digital representation of the patient's atria or ventricles;
identifying, in the digital representation, ablation targets that need to be ablated to terminate determined arrhythmias;
executing, in the digital representation, a mock-up of a clinical ablation procedure of the patient to determine the electrical response of the patient's heart to ablating the ablation targets, and to determine whether the heart continues to generate new arrhythmias post-procedure; and
generating a final set of ablation targets based on the mock-up of the clinical ablation procedure.

2. The computer implemented method of claim 1, further comprising importing, as part of an ablation procedure of the patient, the final set of ablation targets together with a number of anatomical landmarks from the digital representation into a clinical three-dimensional electroanatomical mapping system in a procedure room of an ablation procedure.

3. The computer implemented method of claim 2, further comprising registering the imported final set of ablation targets and the imported landmarks to a heart coordinate system of the patient in the clinical three-dimensional electroanatomical mapping system in the operating room during the ablation procedure.

4. The computer implemented method of claim 1, further comprising displaying the generated final set of ablation targets overlaid over an image of the patient's heart in a clinical electroanatomical mapping system in an operating room during an ablation procedure, and navigating an ablation catheter to the final ablation targets.

5. The computer implemented method of claim 1, wherein the patient imaging data comprises computed tomography (CT) data.

6. The computer implemented method of claim 5, wherein the CT data comprises three-dimensional CT data.

7. The computer implemented method of claim 1, wherein the adipose tissue is one of infiltrating the atrial or ventricular wall, or is epicardial, or pericardial tissue.

8. The computer implemented method of claim 1, wherein the adipose tissue is in combination with fibrosis tissue.

9. The computer implemented method of claim 1, wherein the generating a digital representation of electrical functioning of atria or ventricles of the patient's heart is further based on clinical or experimental data for a regional electrical behavior of cardiac tissue in the presence of adipose tissue.

10. The computer implemented method of claim 1, wherein the ablation procedure comprises one of endocardial, epicardial or intramural needle ablation to access endocardial, epicardial or intramyocardial ablation targets.

11. The computer implemented method of claim 1, wherein when the digital representation continues to generate arrhythmias after ablation of predicted ablation targets in the mock-up of the clinical procedure, determining any new arrhythmias which arise in the ablated digital representation of the patient's atria or ventricles with adipose tissue, and wherein when the new arrhythmias arise, generating additional ablation targets, and adding the additional ablation targets to a set of initial ablation targets.

12. The computer implemented method of claim 11, wherein the determining whether any new arrhythmias arise, and the generating additional ablation targets is repeated, until no new arrhythmias are generated, and the final set of ablation targets is then generated.

13. The computer implemented method of claim 1, wherein the determining whether any new arrhythmias arise comprises delivering pacing to a number of pacing locations of the digital representation of the patient's atria or ventricles.

14. The computer implemented method of claim 1, wherein the generating a digital representation comprises creating a finite element mesh using the imaging data of the patient's heart that reveals the presence of adipose tissue, the finite element mesh comprising a plurality of volume elements, wherein the volume elements each represent a volume having an edge length in a range of about 300-400 microns.

15. The computer implemented method of claim 14, wherein a number of the volume elements is greater than one million.

16. The computer implemented method of claim 15, wherein the number of the volume elements is greater than two million.

17. The computer implemented method of claim 14, where simulations performed with the heart model involve solving a differential equation representing electrical current propagation, together with the system of equations representing cell electrical activity, at each node at the finite element mesh.

18. A system for guiding ablation of atrial or ventricular arrhythmia in a patient's heart, comprising a data processor configured with computer-executable code, the computer-executable code comprising instructions that, when executed by said data processor, causes said data processor to:
generate a digital representation of electrical functioning of atria or ventricles of the patient's heart based on imaging data of the patient's heart that reveals the presence of adipose tissue;
determine any arrhythmias arising in the presence of the adipose tissue in the digital representation of the patient's atria or ventricles;
identify, in the digital representation, ablation targets that need to be ablated to terminate determined arrhythmias;
execute, in the digital representation, a mock-up of a clinical ablation procedure of the patient to determine the electrical response of the patient's heart to ablating the ablation targets, and to determine whether the heart continues to generate new arrhythmias post-procedure; and
generate a final set of ablation targets based on the mock-up of the clinical ablation procedure.

19. The system of claim 18, said computer-executable code further comprising instructions that, when executed by said data processor, causes said data processor to:
import, as part of an ablation procedure of the patient, the final set of ablation targets together with a number of anatomical landmarks from the digital representation into a clinical three-dimensional electroanatomical mapping system in a procedure room of an ablation procedure.

20. The system of claim 18, wherein the patient imaging data comprises computed tomography (CT) data.

21. The system of claim 18, wherein the CT data comprises three-dimensional CT data.

22. The system of claim 18, wherein the adipose tissue is one of infiltrating the atrial or ventricular wall, or is epicardial, or pericardial tissue.

23. The system of claim 18, wherein the adipose tissue is in combination with fibrosis tissue.

24. The system of claim 18, said computer-executable code further comprising instructions that, when executed by said data processor, causes said data processor to:
wherein the generating a digital representation of electrical functioning of atria or ventricles of the patient's heart is further based on clinical or experimental data for a regional electrical behavior of cardiac tissue in the presence of adipose tissue.

25. The system of claim 18, wherein the ablation procedure comprises one of endocardial, epicardial or intramural needle ablation to access intramyocardial ablation targets.

26. The system of claim 18, said computer-executable code further comprising instructions that, when executed by said data processor, causes said data processor to:
wherein when the digital representation continues to generate arrhythmias after ablation of predicted ablation targets in the mock-up of the clinical procedure, determine any new arrhythmias which arise in the ablated digital representation of the patient's atria or ventricles with adipose tissue, and wherein when the new arrhythmias arise, generate additional ablation targets, and add the additional ablation targets to a set of initial ablation targets.

27. The system of claim 26, said computer-executable code further comprising instructions that, when executed by said data processor, causes said data processor to:
wherein the determining whether any new arrhythmias arise, and the generating additional ablation targets is repeated, until no new arrhythmias are generated, and the final set of ablation targets is then generated.

28. The system of claim 18, wherein the determining whether any new arrhythmias arise comprises delivering pacing to a number of pacing locations of the digital representation of the patient's atria or ventricles.

29. The system of claim 18, wherein the generating a digital representation comprises creating a finite element mesh using the imaging data of the patient's heart that reveals the presence of adipose tissue, the finite element mesh comprising a plurality of volume elements, wherein the volume elements each represent a volume having an edge length in a range of about 300-400 microns.

30. The system of claim 29, wherein a number of the volume elements is greater than one million.

31. The system of claim 30, wherein the number of the volume elements is greater than two million.
